(19) United States
(12) Patent Application Publication
(10) Pub. No.: US 2023/0242547 A1
(43) Pub. Date: Aug. 3, 2023

(54) PROCESSES FOR PREPARING NOR-OPIOID COMPOUNDS AND OPIOID ANTAGONISTS BY ELECTROCHEMICAL N-DEMETHYLATION

(71) Applicants: RESEARCH CENTER PHARMACEUTICAL ENGINEERING GMBH, Graz (AT); KARL-FRANZENS-UNIVERSITÄT GRAZ, Graz (AT)

(72) Inventors: Gabriel GLOTZ, Graz (AT); David CANTILLO NIEVES, Graz (AT); Christian Oliver KAPPE, Graz (AT)

(21) Appl. No.: 18/001,219

(22) PCT Filed: May 10, 2021
(86) PCT No.: PCT/EP2021/062298

§ 371 Date: Dec. 8, 2022

(30) Foreign Application Priority Data
Jun. 10, 2020 (DE) 10 2020 115 418.6

(51) Int. Cl. C07D 491/08; C25B 3/05

(52) U.S. Cl. CPC C07D 491/08; C25B 3/05

(57) ABSTRACT

The present disclosure relates to a process for preparing a nor-opioid compound wherein an opioid precursor compound is electrochemically N-demethylated. The present disclosure further relates to a process for preparing an opioid antagonist compound, wherein an opioid precursor compound is electrochemically N-demethylated and the thus obtained nor-opioid compound is alkylated again at its secondary amine functional group.

This application is the U.S. National Phase of International Application No. PCT/EP2021/062298 filed 10 May 2021 which designated the U.S. and claims priority to German Patent Application No. 10 2020 115 418.6 filed 10 Jun. 2020, the entire contents of each of which are hereby incorporated herein by reference.

### FIELD OF THE DISCLOSURE

Embodiments of the present disclosure relate to a process for preparing a nor-opioid compound from an opioid precursor compound by N-demethylation and further relates to a process for preparing an opioid antagonist compound from an opioid precursor compound via the nor-opioid compound.

### BACKGROUND

Most naturally occurring morphinan alkaloids, such as morphine, codeine, oripavine or thebaine, as well opioid analgesics such as oxycodone contain a tertiary N-methylamine group in their structural formula. Substitution of the N-methyl group by another moiety has a significant impact in their pharmacological properties. Indeed, many semi-synthetic opioid antagonists (e.g., naltrexone, naloxone, and nalbuphine) are prepared by attaching a different alkyl group to the nitrogen. This is accomplished by a process consisting of the N-demethylation of the opioid precursors followed by alkylation of the nor-derivative with an alkyl bromide, as illustrated in FIG. 1A (U. Rinner, T. Hudlicky, Synthesis of Morphine Alkaloids and Derivatives. In: Alkaloid Synthesis (Ed.: H. J. Knölker). Topics in Current Chemistry, vol 309. Springer, Berlin, Heidelberg, 2011, pp 33-66; S. Thavaneswaran, K. McCamley, P. J. Scammells, Nat. Prod. Commun. 2006, 1, 885-897).

Selective removal of the N-methyl group from 14-hydroxy morphinan precursors can be challenging. This step is often carried out using excess amounts of harmful electrophilic reagents like cyanogen bromide (via the von Braun reaction) (S. Hosztafi, C. Simon, S. Makleit, Synth. Commun. 1992, 22, 1673-1682; H. Yu, T. Prisinzano, C. M. Dersch, J. Marcus, R. B. Rothman, A. E. Jacobson, K. C. Ricea, Bioorg. Med. Chem. Lett. 2002, 12, 165-168; B. R. Selfridge, X. Wang, Y. Zhang, H. Yin, P. M. Grace, L. R. Watkins, A. E. Jacobson, K. C. Rice, J. Med. Chem. 2015, 58, 5038-5052; J. Marton, S. Miklòs, S. Hosztafi, S. Makleit, Synth. Commun. 1995, 25, 829-848; H. S. Park, H. Y. Lee, Y. H. Kim, J. K. Park, E. E. Zvartauc, H. Lee, Bioorg. Med. Chem. Lett. 2006, 16, 3609-3613) or chloroalkyl formates (P. X. Wang, T. Jiang, G. L. Cantrell, D. W. Berberich, B. N. Trawick, T. Osiek, S. Liao, F. W. Moser, J. P. McClurg (Mallinckrodt Inc.), cf. also US 20090156818A1; P. X. Wang, T. Jiang, G. L. Cantrell, D. W. Berberich, B. N. Trawick, S. Liao (Mallinckrodt Inc.), cf. also US 20090156820A1; S. Hosztafi, S. Makleit, Synth. Commun., 1994, 24, 3031-3045; A. Ninan, M. Sainsbury, Tetrahedron, 1992, 48, 6709-6716). The combination of stoichiometric amounts of peroxides and acylating agents (classical Polonovski reaction) or metal reductants (non-classical Polonovski reaction) has also been applied (M. Ann, A. Endoma-Arias, D. P. Cox, T. Hudlicky, Adv. Synth. Catal., 2013, 355, 1869-1873; G. Kok, T. D. Asten and P. J. Scammells, Adv. Synth. Catal., 2009, 351, 283-286; Z. Dong, P. J. Scammells, J. Org. Chem., 2007, 72, 9881-9885; T. Rosenau, A. Hofinger, A. Potthast, P. Kosma, Org. Lett., 2004, 6, 541-544; D. D. D. Pham, G. F. Kelso, Y. Yang, M. T. W. Hearn, Green Chem. 2012, 14, 1189-1195; D. D. D. Pham, G. F. Kelso, Y. Yang, M. T. W. Hearn, Green Chem. 2014, 16, 1399-1409; Y. Li, L. Ma, F. Jia, Z. Li, J. Org. Chem. 2013, 78, 5638-5646).

More benign alternatives have been actively investigated during the past two decades, including palladium catalyzed (R. J. Carroll, H. Leisch, E. Scocchera, T. Hudlicky, D. P. Cox, Adv. Synth. Catal., 2008, 350, 2984-2992; A. Machara, L. Werner, M. A. Endoma-Arias, D. P. Cox, T. Hudlicky, Adv. Synth. Catal. 2012, 354, 613-626; A. Machara, D. P. Cox, T. Hudlicky, Adv. Synth. Catal. 2012, 354, 2713-2718; B. Gutmann, U. Weigl, D. P. Cox, C. O. Kappe, Chem. Eur. J. 2016, 22, 10393-10398; B. Gutmann, P. Elsner, D. P. Cox, U. Weigl, D. M. Roberge, C. O. Kappe, ACS Sust. Chem. Eng. 2016, 4, 6048-6061; B. Gutmann, D. Cantillo, U. Weigl, D. P. Cox, C. O. Kappe, Eur. J. Org. Chem. 2017, 914-927; A. Mata, D. Cantillo, C. O. Kappe, Eur. J. Org. Chem. 2017, 24, 6505-6510; WO 2017/184979 A1; WO 2017/185004 A1) and photochemical (J. A. Ripper, E. R. Tiekink, P. J. Scammells, Bioorg. Med. Chem. Lett. 2001, 11, 443-445; Y. Chen, G. Glotz, D. Cantillo, Chem. Eur. J. 2020, 26, 2973-2979) aerobic oxidations as well as chemoenzymatic procedures (M. M. Augustin, J. M. Augustin, J. R. Brock, T. M. Kutchan, Nat. Sustain. 2019, 2, 465-474). However, these methods have not been adopted by industry.

Thus, there might be a demand for further improvements in the N-demethylation process of an opioid precursor compound that addresses and overcomes the disadvantages and drawbacks discussed above.

There may be a need to provide a process for preparing a nor-opioid compound from an opioid precursor compound by N-demethylation (in the following also referred to as “N-demethylation process”) that is highly convenient, sustainable and cost-efficient, in particular a one-pot process that does not require stoichiometric amounts of hazardous electrophilic reagents or catalysts and may be carried out using benign solvents and under mild conditions. There may be also a need to provide a process for preparing an opioid antagonist compound from an opioid precursor compound via the thus prepared nor-opioid compound.

### SUMMARY OF THE DISCLOSURE

The present inventors have made diligent studies and have found that the N-demethylation of an opioid precursor compound can be achieved electrochemically, in particular by an electrolytic (more specifically anodic) oxidation of the N-methyl group, in a reagent-free and catalyst-free manner and may provide the target compounds in good yields. Without wishing to be bound to any theory, the inventors assume that the N-methyl group may be anodically oxidized to a corresponding iminium cation in a 2-electron process. The inventors further assume that the ensuing iminium cation rapidly undergoes cyclization with the vicinal 14-hydroxy group or a substituent transfer from its substituted derivative occurs, resulting in intermediates (such as oxazolidine intermediates and 14-O-substituent transfer intermediates, respectively) that can be readily hydrolyzed to the target nor-opioid compounds (as illustrated in FIG. 1B), which may subsequently be alkylated again at the nitrogen to yield the target opioid antagonist compounds.

Accordingly, an exemplary embodiment relates to a process for preparing a compound of Formula (I) (herein also referred to as “nor-opioid compound” or simply as “nor-opioid”)

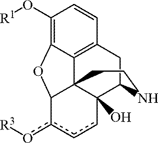

wherein
each

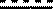

represents a single or double bond, provided that two double bonds are not adjacent to each other;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group;
R³ is selected from the group consisting of C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group or is absent;
wherein one or more hydrogen atoms on the R¹ and R³ groups may be replaced with F and/or Cl;
comprising the steps of
providing a compound of Formula (II) (herein also referred to as “opioid precursor compound” or simply as “opioid precursor”)

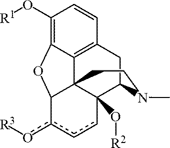

wherein
R¹, R³ and

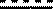

are as defined above; and
R² is selected from the group consisting of H, C(O)R⁶, S(O)R⁶,SO₂R⁶, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅₋₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl; and
electrochemically demethylating the compound of Formula (II).

Another exemplary embodiment relates to a process for preparing a compound of Formula (V) (herein also referred to as “opioid antagonist compound” or simply as “opioid antagonist”)

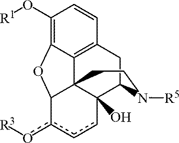

wherein
each

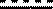

represents a single or double bond, provided that two double bonds are not adjacent to each other;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group;
R³ is selected from the group consisting of C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group or is absent;
R⁵ is selected from the group consisting of C₁₋₁₀ alkyl, C₂₋₁₀ alkenyl, C₆₋₁₀ aryl, C₃₋₁₀ cycloalkyl, C₃₋₁₀ cycloalkenyl, C₁₋₁₀ alkylene-C₆₋₁₀ aryl, C₁₋₁₀ alkylene-C₃₋₁₀ cycloalkyl;
wherein one or more hydrogen atoms on the R¹, R³ and R⁵ groups may be replaced with F and/or Cl;
comprising the steps of
providing a compound of Formula (II)

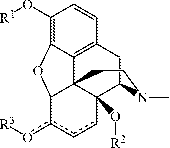

wherein
R¹, R³ and

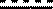

are as defined above; and
R² is selected from the group consisting of H, C(O)R⁶, S(O)R⁶,SO₂R⁶, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅₋₁₀ heteroaryl, each of the groups being unsubstituted
or substituted with one or more substituents independently selected from C₁₋₄ alkyl, O-C₁₋₄ alkyl, halogen, CN, NO₂, C₆₋₁₀ aryl and O-C₆₋₁₀ aryl;
electrochemically demethylating the compound of Formula (II) to yield a compound of Formula (I)

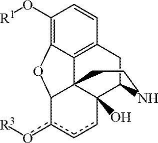

wherein R¹, R³ and

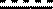

are as defined above; and
subsequently reacting the compound of Formula (I) with a compound of Formula (VI)

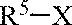

wherein R⁵ is as defined above and X represents a leaving group (counteranion), in the presence of a base.

Other objects and many of the attendant advantages of embodiments of the present disclosure will be readily appreciated and become better understood by reference to the following detailed description of embodiments and examples and the accompanying drawings .

### BRIEF DESCRIPTION OF THE DRAWINGS

FIG. 1 illustrates exemplary embodiments of reaction schemes of (A) a general synthesis of opioid antagonists from opioid precursors via a nor-opioid derivative by a sequence of N-demethylation and alkylation, (B) conventional processes for preparing a nor-opioid derivative according to the prior art, and (C) the novel electrochemical approach for preparing a nor-opioid derivative according to an embodiment of the present disclosure.

FIG. 2 shows an exemplary embodiment of a setup for a flow electrolysis for an N-demethylation process according to an embodiment of the present disclosure.

### DETAILLED DESCRIPTION OF EXAMPLARY EMBODIMENTS

Hereinafter, details of the present disclosure and other features and advantages thereof will be described. However, the present disclosure is not limited to the following specific descriptions, but they are rather for illustrative purposes only.

It should be noted that features described in connection with one exemplary embodiment or exemplary aspect may be combined with any other exemplary embodiment or exemplary aspect, in particular features described with any exemplary embodiment of an N-demethylation process may be combined with any further exemplary embodiment of an N-demethylation process as well as with any exemplary embodiment of process for preparing an opioid antagonist and vice versa, unless specifically stated otherwise.

Where an indefinite or definite article is used when referring to a singular term, such as “a”, “an” or “the”, a plural of that term is also included and vice versa, unless specifically stated otherwise, whereas the word “one” or the number “1”, as used herein, typically means “just one” or “exactly one”.

The expression “comprising”, as used herein, includes not only the meaning of “comprising”, “including” or “containing”, but also encompasses “consisting essentially of” and “consisting of”.

In a first aspect, an exemplary embodiment relates to a (one-pot) process for preparing a compound of Formula (I)

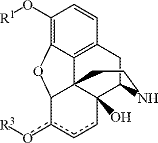

wherein
each

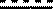

represents a single or double bond, provided that two double bonds are not adjacent to each other;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group;
R³ is selected from the group consisting of C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group or is absent;
wherein one or more hydrogen atoms on the R¹ and R³ groups may be replaced with F and/or Cl;
comprising the steps of
providing a compound of Formula (II)

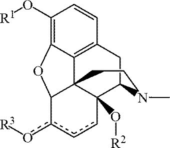

wherein
R¹, R³ and

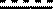

are as defined above; and
R² is selected from the group consisting of H, C(O)R⁶, S(O)R⁶,SO₂R⁶, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅₋₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl; and
electrochemically demethylating the compound of Formula (II).

The term “alkyl”, as used herein, refers to, whether it is used alone or as part of another group, straight- or branched-chain, saturated alkyl groups. The term “C₁₋₁₀ alkyl” means an alkyl group having 1, 2, 3, 4, 5, 6, 7, 8, 9 or 10 carbon atoms. In some embodiments, one or more, including all of the available hydrogen atoms in the alkyl groups may be replaced with a halogen, such as F and/or Cl.

The term “aryl”, as used herein, refers to cyclic groups that contain at least one aromatic ring. The aryl group may contain 6, 9 or 10 atoms, such as phenyl, naphthyl or indanyl. In some embodiments, one or more, including all of the available hydrogen atoms in the aryl groups may be replaced with a halogen, such as F and/or Cl.

The term “cycloalkyl”, as used herein, refers to, whether it is used alone or as part of another group, cyclic, saturated alkyl groups. The term “C₃₋₁₀ cycloalkyl” means a cycloalkyl group having 3, 4, 5, 6, 7, 8, 9 or 10 carbon atoms. In some embodiments, one or more of the hydrogen atoms in the cycloalkyl groups may be replaced with a halogen, such as F and/or Cl.

The term “alkylene”, as used herein, refers to, whether alone or as part of another group, an alkyl group that is bivalent; i.e. that is substituted on two ends with another group. The term “C₁₋₁₀ alkylene” means an alkylene group having 1, 2, 3, 4, 5, 6, 7, 8, 9 or 10 carbon atoms. In some embodiments, one or more, including all of the available hydrogen atoms in the alkylene groups may be replaced with a halogen, such as F and/or Cl.

The term “protecting group”, as used herein, refers to a chemical moiety which protects or masks a reactive portion of a molecule to prevent side reactions in those reactive portions of the molecule, while reacting a different portion of the molecule. Thus, a protecting group may be introduced into a molecule by chemical modification of a functional group so as to achieve chemoselectivity in a subsequent chemical reaction. After the reaction is completed, the protecting group can be removed under conditions that do not degrade or decompose the remaining portions of the molecule. The selection of a suitable protecting group can be appropriately made by a person skilled in the art. Examples of suitable protecting groups include, but are not limited to acetyl, benzoyl and silyl ethers, such as t-butyl-dimethylsilyl (TBDMS) or trimethylsilyl (TMS). In an embodiment, it might be advantageous that R¹ in the opioid precursor compound of Formula (II) is a protecting group so as to efficiently avoid an undesired oxidation of the phenolic moiety (i.e. if R¹ = H) during the step of electrochemically demethylating the opioid precursor compound, in particular in case of an anodic oxidation thereof.

The term “heterocycloalkyl″”, as used herein, refers to, whether it is used alone or as part of another group, cyclic, saturated alkyl groups containing at least one heteroatom, such as N, O and/or S. The term “C₃₋₁₀ heterocycloalkyl” means a heterocycloalkyl group having 3, 4, 5, 6, 7, 8, 9 or 10 atoms including carbon atoms, in which at least one atom is a heteroatom, such as N, O and/or S. In some embodiments, one or more, including all of the available hydrogen atoms in the heterocycloalkyl groups may be replaced with a halogen, such as F and/or Cl.

The term “cycloalkenyl”, as used herein, refers to, whether it is used alone or as part of another group, cyclic, unsaturated alkyl groups. The term “C₃₋₁₀ cycloalkenyl” means a cycloalkenyl group having 3, 4, 5, 6, 7, 8, 9 or 10 carbon atoms and at least one double bond. In some embodiments, one or more, including all of the available hydrogen atoms in the cycloalkenyl groups may be replaced with a halogen, such as F and/or Cl.

The term “alkenyl”, as used herein, refers to, whether it is used alone or as part of another group, straight- or branched-chain, unsaturated alkenyl groups. The term “C₂₋₁₀ alkenyl” means an alkenyl group having 2, 3, 4, 5, 6, 7, 8, 9 or 10 carbon atoms and at least one double bond. In some embodiments, one or more, including all of the available hydrogen atoms in the alkenyl groups may be replaced with a halogen, such as F and/or Cl.

The term “heteroaryl”, as used herein, refers to cyclic groups that contain at least one aromatic ring and at least one heteroatom, such as N, O and/or S.

The term “C₅₋₁₀ heteroaryl” means an aryl group having 5, 6, 7, 8, 9 or 10 atoms including carbon atoms, in which at least one atom is a heteroatom, such as N, O and/or S. In some embodiments, one or more, including all of the available hydrogen atoms in the heteroaryl groups may be replaced with a halogen, such as F and/or Cl.

In an embodiment, R² is at least one of H or an acyl group, such as C₁₋₁₀ acyl. The term “acyl”, as used herein, refers to, whether it is used alone or as part of another group, a straight or branched, saturated alkyl chain bound at a carbonyl (—C(O)—) group. The term C₁₋₁₀ acyl means an acyl group having 2, 3, 4, 5, 6, 7, 8, 9, 10 or 11 carbon atoms (i.e. —C(O)—C₁₋₁₀ alkyl). In some embodiments, one or more, including all of the available hydrogen atoms in the acyl groups may be replaced with a halogen, such as F and/or Cl, and thus may include, for example trifluoroacetyl.

In an embodiment, the nor-opioid compound is a compound of Formula (Ia) depicted below and the opioid precursor compound is a compound of Formula (IIa) depicted below. In this embodiment, R³ in the compounds of Formulas (I) and (II) is absent.

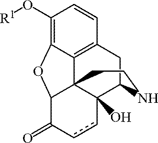

wherein

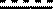

represents a single or double bond;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group, wherein one or more hydrogen atoms on the R¹ groups may be replaced with F and/or Cl.

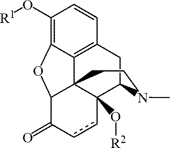

wherein
R¹ and

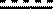

are as defined above; and
R² is selected from the group consisting of H, C(O)R⁶, S(O)R⁶,SO₂R⁶, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅₋₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl.

In an embodiment, the compound of Formula (II) is selected from the group consisting of oxymorphone, oxycodone, 14-hydroxycodeinone, 14-hydroxymorphinone, oxymorphone-3,14-diacetate, 14-hydroxymorphinone-3,14-diacetate, 14-acetyloxycodone, 14-hydroxycodeinone O-acetyl ester and 6-oxycodol. The chemical structures of some of these specific opioid precursor compound are depicted below:

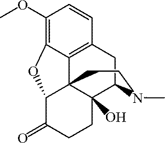

Oxycodone (1a)

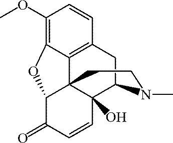

14-Hydroxycodeinone

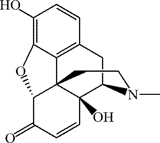 [SMILES: CN1CC[C@]23c4c5ccc(O)c4O[C@H]2C(=O)C=C[C@@]3(O)[C@H]1C5]

14-Hydroxymorphinone

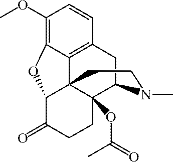

14-acetyloxycodone

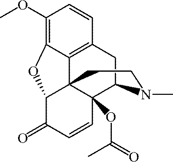

14-hydroxycodeinone O-acetyl ester

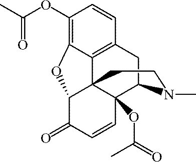 [SMILES: CC(=O)Oc1ccc2c3c1O[C@H]1C(=O)C=C[C@@]4(OC(C)=O)[C@@H](C2)N(C)CC[C@]314]

14-hydroxymorphinone-3,14-diacetate

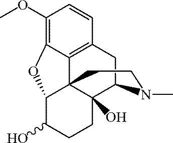

6-Oxycodol (1e)

The opioid precursor compound of Formula (II) may be provided or prepared by conventional synthesis methods as known to a person skilled in the art. Examples of suitable methods are described for instance in A. Mata, D. Cantillo, C. O. Kappe, Eur. J. Org. Chem. 2017, 24, 6505-6510; A. Machara, M. A. A. Endoma-Arias, I. Cisařova, D. P. Cox, T. Hudlicky, Synthesis 2016, 48, 1803-1813; C.-Y. Cheng, L.-W. Hsin, Y.-P. Lin, P.-L. Tao, T.-T. Jong, Bioorg. Med. Chem. 1996, 4, 73-80; F. I. Carroll, C. G. Moreland, G. A. Brine, J. A. Kepler, J. Org. Chem. 1976, 41, 6, 996-1001; and A. C. Currie, G. T. Newbold, F. S. Spring, J. Chem. Soc. 1961, 4693-4700.

In an embodiment, the step of electrochemically demethylating the compound of Formula (II) comprises an electrolytic oxidation of the tertiary N-methylamine functional group of the compound of Formula (II) and subsequently treating (reacting, hydrolyzing) a thus obtained intermediate with an acid (i.e. hydrolyzing under acidic conditions) to yield the compound of Formula (I). Thus, the tertiary N-methylamine functional group of the compound of Formula (II) may be electrolytically (in particular anodically) oxidized to yield an intermediate, such as an oxazolidine intermediate or a 14-O-substituent transfer intermediate to be described in further detail below, and directly (i.e. without any isolation or purification thereof) or indirectly (i.e. with an isolation and/or purification thereof) converted into the target nor-opioid compound of Formula (I) by hydrolysis, which may be achieved for instance by treating the intermediate with an acid. It may be advantageous to treat the intermediate with an acid at an elevated temperature, for instance under reflux. In particular, the conversion of the opioid precursor compound of Formula (II) to the nor-opioid compound of Formula (I) may be carried as a one-pot process.

In an embodiment, the intermediate may comprise a compound of Formula (III) (herein also referred to as “oxazolidine intermediate”) or a compound of Formula (IV) (herein also referred to as “14-O-substituent transfer intermediate”):

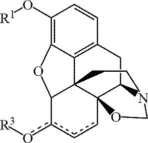

wherein R¹, R³ and

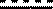

are as defined above;

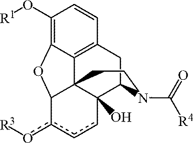

wherein R¹, R³ and

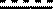

are as defined above and R⁴ is selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅₋₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl.

An oxazolidine intermediate may in particular be formed if R² in the opioid precursor compound of Formula (II) is H, whereas a 14-O-substituent transfer intermediate may in particular be formed if R² in the opioid precursor compound of Formula (II) is a group other than H, more specifically C(O)R⁶, such as an acyl group. In some embodiments, the 14-O-substituent transfer intermediate may therefore also be referred to as “acyl transfer intermediate”.

In an embodiment, the step of electrochemically demethylating the compound of Formula (II) comprises an electrolytic oxidation of the tertiary N-methylamine functional group of the compound of Formula (II) by means of an electrolytic unit (such as an electrolytic cell) comprising at least two electrodes and an electrolyte.

In an embodiment, the electrolytic unit comprises an anode and a cathode, wherein the tertiary N-methylamine functional group of the compound of Formula (II) is electrolytically oxidized at the anode.

In an embodiment, the anode comprises at least one of the group consisting of a carbon-containing material, such as graphite, reticulated vitreous carbon, glassy carbon, carbon felt, or boron-doped diamond, and platinum. In particular, graphite and impervious graphite have proven particularly suitable and at the same time inexpensive materials for the anode, but also platinum and other carbon-containing materials have proven suitable materials for the anode.

In an embodiment, the cathode comprises at least one of the group consisting of an iron-containing material, in particular stainless steel, a nickel-containing material, platinum, lead, mercury and a carbon-containing material, such as graphite, reticulated vitreous carbon, glassy carbon, carbon felt, or boron-doped diamond. In particular, stainless steel has proven a particularly suitable and at the same time inexpensive material for the cathode, but also nickel and platinum have proven suitable materials for the cathode.

In an embodiment, the electrolyte is selected from the group consisting of a quaternary ammonium salt, a lithium salt, a sodium salt, a potassium salt and mixtures or combinations thereof. Suitable examples of the quaternary ammonium salt include tetraalkylammonium (such as tetraethylammonium or tetrabutylammonium) salts having tetrafluoroborate or hexafluorophosphate anions, such as tetraethylammonium tetrafluoroborate (Et₄NBF₄), tetrabutylammonium tetrafluoroborate (nBu₄NBF₄) and tetrabutylammonium hexafluorophosphate (nBu₄NPF₆). Suitable examples of potassium salts include potassium acetate (KOAc). Suitable examples of lithium salts include lithium perchlorate (LiClO₄), lithium tetrafluoroborate (LiBF₄)and lithium hexafluorophosphate (LiPF₆) and suitable examples of sodium salts include sodium perchlorate (NaClO₄), sodium tetrafluoroborate (NaBF₄) and sodium hexafluorophosphate (NaPF₆). In particular, quaternary ammonium and potassium salts have proven particularly suitable for solving the object of the present disclosure. Potassium acetate (KOAc) has shown particularly suitable in terms of an improved efficiency (yield and selectivity) of the N-demethylation process.

In an embodiment, the electrolytic unit further comprises a solvent. While not excluded, it is not required for the N-demethylation process according to the present disclosure that the solvent is anhydrous, which contributes to a convenient and cost-effective process.

In particular, it may be advantageous to use a protic solvent for the N-demethylation process according to the present disclosure. The term “protic solvent”, as used herein, refers to a solvent that is capable of donating protons (H⁺). By the addition of a protic solvent, a source of protons for a concurrent cathodic reduction may be provided. Without wishing to be bound to any theory, the inventors assume that although two protons are released during the formation of an iminium cation intermediate, a protic solvent may facilitate their transport and enhance the cathodic reduction. As a result, efficiency of the N-demethylation process may be improved.

In an embodiment, the solvent is selected from the group consisting of acetonitrile, dimethylformamide, dimethylacetamide, methanol, ethanol, n-propanol, isopropanol, hexafluoroisopropanol (HFIP), trichloromethane (chloroform), dichloromethane, tetrahydrofuran, methyltetrahydrofuran, acetone and mixtures or combinations thereof. It may be advantageous to use mixtures or combinations of these solvents. In particular a combination of acetonitrile (MeCN) and methanol (MeOH), for instance in a volume ratio MeCN/MeOH of from 1:10 to 10:1, such as 4:1, has proven particularly suitable for solving the object of the present disclosure. In particular, ethanol as the solvent, preferably in combination with potassium acetate (KOAc) as the electrolyte, has shown particularly suitable in terms of an improved efficiency (yield and selectivity) of the N-demethylation process.

In an embodiment, the step of electrochemically demethylating the compound of Formula (II) may be carried out at room temperature, but may also be carried out in a temperature range of from 5 to 50° C., such as from 10 to 40° C.

In an embodiment, the step of electrochemically demethylating the compound of Formula (II) may be carried out at ambient pressure, but may also be carried out under a pressure range of from 0.1 to 20 bar. Ambient pressure has shown particularly suitable in terms of an improved efficiency (yield and selectivity) of the N-demethylation process.

The duration of the step of electrochemically demethylating the compound of Formula (II) is not particularly limited and may be appropriately adjusted by a person skilled in the art, for instance by monitoring the reaction and thereby determining the completion of the conversion.

The (gas) atmosphere in the electrolytic unit while carrying out the step of electrochemically demethylating the compound of Formula (II) is not particularly limited and may be appropriately selected by a person skilled in the art. While not excluded, an inert atmosphere is not required for the N-demethylation process according to the disclosure, which contributes to a convenient and cost-effective process.

In an embodiment, the step of electrochemically demethylating the compound of Formula (II) may be carried out at concentrations in the range from 0.01 to 2 M. Concentrations in the range from of 0.05 to 0.2 M have shown particularly suitable in terms of an improved efficiency (yield and selectivity) of the N-demethylation process.

In an embodiment, the molar ratio between the compound of Formula (II) and the electrolyte may range from 10:1 to 1:10. Substrate/electrolyte molar ratios in the range from 2:1 to 1:2 have shown particularly suitable in terms of an improved efficiency (yield and selectivity) of the N-demethylation process.

In an embodiment, the step of electrochemically demethylating the compound of Formula (II) comprises an electrolytic oxidation of the tertiary N-methylamine functional group of the compound of Formula (II) under constant current (galvanostatic) conditions, but may also be carried out under constant potential (potentiostatic) conditions. Current densities from 1 mA/cm² to 300 mA/cm² may be utilized under constant current. Current densities in the range of 2 mA/cm² to 20 mA/cm² have proven particularly suitable for solving the object of the present disclosure. Cell voltages from 1 V to 30 V may be utilized. Cell voltages in the range of 2 to 5 V have proven particularly suitable for solving the object of the present disclosure.

In an embodiment, the step of electrochemically demethylating the compound of Formula (II) comprises an electrolytic oxidation of the tertiary N-methylamine functional group of the compound of Formula (II) in a batchwise (i.e. discontinuous) manner.

In an alternative embodiment, the step of electrochemically demethylating the compound of Formula (II) comprises an electrolytic oxidation of the tertiary N-methylamine functional group of the compound of Formula (II) in a continuous manner, in particular using a flow cell, such as a flow electrolysis cell. A suitable flow electrolysis cell is described for instance in A. A. Folgueiras-Amador, K. Philipps, S. Guilbaud, J. Poelakker, T. Wirth, Angew. Chem. Int. Ed. 2017, 56, 15446-15450; D. Pletcher, R. A. Green, R. C. D. Brown, Chem. Rev. 2018, 118, 4573-4591; and T. Noël, Y. Cao, G. Laudadio, Acc. Chem. Res. 2019, 52, 2858-2869.

In an embodiment, the acid is selected from the group consisting of hydrochloric acid, acetic acid and sulfuric acid.

In a second aspect, another exemplary embodiment relates to process for preparing a compound of Formula (V)

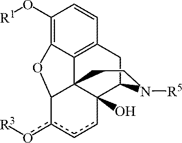

wherein
each

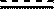

represents a single or double bond, provided that two double bonds are not adjacent to each other;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group;
R³ is selected from the group consisting of C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group or is absent;
R⁵ is selected from the group consisting of C₁₋₁₀ alkyl, C₂₋₁₀ alkenyl, C₆₋₁₀ aryl, C₃₋₁₀ cycloalkyl, C₃₋₁₀ cycloalkenyl, C₁₋₁₀ alkylene-C₆₋₁₀ aryl, C₁₋₁₀ alkylene-C₃₋₁₀ cycloalkyl;
wherein one or more hydrogen atoms on the R¹, R³ and R⁵ groups may be replaced with F and/or Cl;
comprising the steps of
providing a compound of Formula (II)

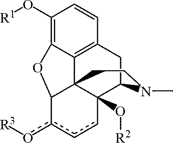

wherein
R¹, R³ and

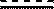

are as defined above; and
R² is selected from the group consisting of H, C(O)R⁶, S(O)R⁶,SO₂R⁶, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅₋₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl;
electrochemically demethylating the compound of Formula (II) to yield a compound of Formula (I)

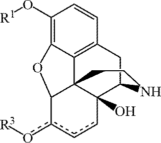

wherein R¹, R³ and

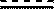

are as defined above; and
subsequently reacting the compound of Formula (I) with a compound of Formula (VI)

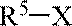

wherein R⁵ is as defined above and X represents a leaving group (counteranion), in the presence of a base.

The compounds of Formulae (I) and (II) as well as the step of electrochemically demethylating the compound of Formula (II) to yield a compound of Formula (I) may in particular be those as described in detail above with regard to the N-demethylation process according to the present disclosure.

In an embodiment, the step of reacting the compound of Formula (I) with a compound of Formula (VI) is carried in a solvent. Suitable examples thereof include dimethylformamide, dimethylacetamide, dimethylsulfoxide and mixtures or combinations thereof.

In an embodiment, the step of reacting the compound of Formula (I) with a compound of Formula (VI) is carried in the presence of a base (i.e. under basic conditions). Suitable examples thereof include sodium carbonate, potassium carbonate, disodium hydrogenphosphate, dipotassium hydrogenphosphate and mixtures or combinations thereof

In an embodiment, the step of reacting the compound of Formula (I) with a compound of Formula (VI) is carried at a temperature in a range of from 50° C. to 100° C., such as from 60° C. to 90° C.

In an embodiment, R⁵ is selected from C₂₋₁₀ alkenyl and C₁₋₁₀ alkylene-C₃₋₁₀ cycloalkyl, in particular from allyl, cyclopropylmethyl and cyclobutylmethyl.

The term “leaving group”, as used herein, refers to a molecular fragment that departs with a pair of electrons in heterolytic bond cleavage. The leaving group may in particular refer to a group that is readily displaceable by a nucleophile, for instance under nucleophilic substitution reaction conditions. In an embodiment, the leaving group corresponds to a counteranion. Examples of suitable leaving groups include for instance halogen (anions) and tosylate, preferably bromide.

In an embodiment, the compound of Formula (VI) is selected from the group consisting of allylbromide, cyclopropylmethyl bromide and cyclobutylmethyl bromide.

In an embodiment, the compound of Formula (V) is selected from the group consisting of naloxone, naltrexone and nalbuphine.

The present disclosure is further described by reference to the accompanying figures and by the following examples, which are solely for the purpose of illustrating specific embodiments and shall not be construed as limiting the scope of the disclosure in any way.

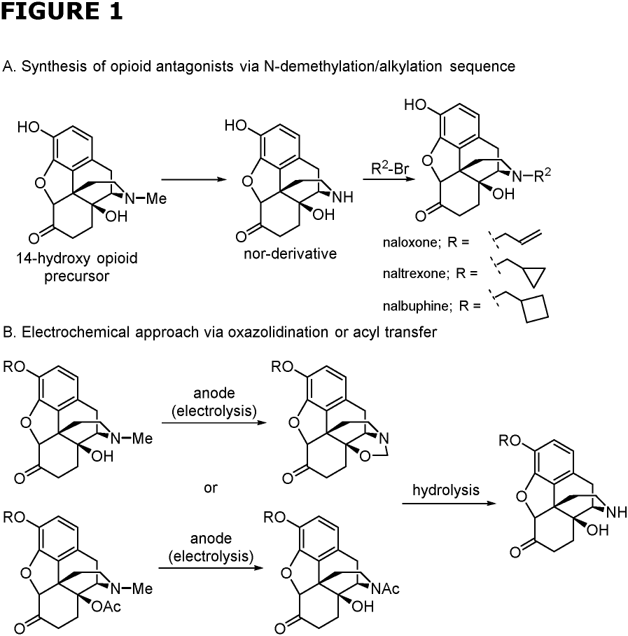

FIG. 1 illustrates exemplary embodiments of various reaction schemes.

FIG. 1A illustrates the general synthesis of opioid antagonists from opioid precursors via a nor-opioid derivative by a sequence of N-demethylation and alkylation.

FIG. 1B illustrates exemplary embodiments of an N-demethylation process according to an embodiment of the present disclosure wherein the N-methylated opioid precursor compound is subjected to an electrolytic oxidation (as illustrated by a power plug) thereby N-demethylating the opioid precursor compound via oxazolidination or acyl transfer to yield the respective oxazolidine and acyl transfer intermediates and the ensuing intermediates are then hydrolyzed by acidic workup to yield the desired nor-opioid compounds. This novel electrochemical approach enables a reagent- and catalyst-free, easily scalable process under mild conditions that provides quantitative yields of the nor-opioid compounds.

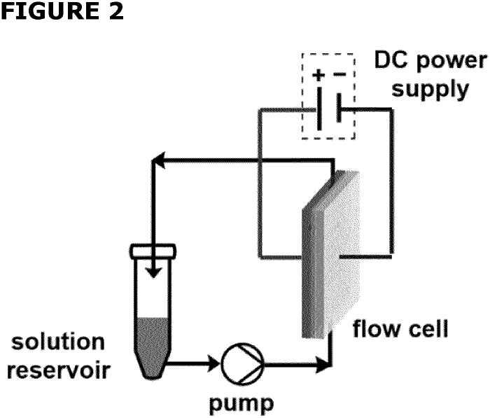

FIG. 2 shows an illustrative embodiment of a setup for a flow electrolysis for an N-demethylation process according to an embodiment of the present disclosure.

The depicted setup for the flow electrolysis comprises a solution reservoir with electrolyte recycle. The reaction mixture is pumped with a Syrris syringe pump through the assembled flow cell, which is powered by a DC power supply. Further details on the experimental procedure for the electrolysis will be given in the context of the Examples below.

The flow cell consists of a parallel plate arrangement with the two electrodes separated e.g. by a 0.3 mm chemically resistant Mylar film incorporating a reaction channel. The contact surface area between the electrodes and the solution is for instance 6.4 cm². The reaction mixture is pumped through the cell using a syringe pump and recirculated at a flow rate of for instance 2 mL/min until the desired amount of charge has been passed. Using an identical reaction mixture as in batch mode and a current of 10 mA, the outcome of the reaction in terms of conversion rate and selectivity was analogous to a batch process. No inert atmosphere or anhydrous solvents is required to perform this transformation. The N-demethylation, that otherwise is generally executed using rather hazardous reagents in stoichiometric quantities, is driven here simply by electricity via inexpensive electrode materials and producing hydrogen as byproduct.

The flow electrolysis cell utilized is based on a typical parallel plates arrangement as described in A. A. Folgueiras-Amador, K. Philipps, S. Guilbaud, J. Poelakker, T. Wirth, Angew. Chem. Int. Ed. 2017, 56, 15446-15450, and D. Pletcher, R. A. Green, R. C. D. Brown, Chem. Rev. 2018, 118, 4573-4591. The two electrode plates are placed facing each other and separated by an interelectrode membrane made of 0.3 mm thick chemically resistant Mylar film, that incorporates a reaction channel. The channel provides a contact surface area of 6.4 cm² between the liquid stream and the electrodes. A graphite plate (IG-15, GTD Graphit Technologie GmbH, 50 × 50 × 3 mm) is utilized as anode and a 304 stainless steel plate (50 × 50 × 1 mm) is used as cathode. To ensure that current cannot flow between the two end plates in case of electrolyte leakage, polyamide bolts are utilized to assemble the cell.

### EXAMPLES

I) Initially, the preparation of various opioid precursor compounds is described.

### 1. Synthesis of Oxycodone (1a)

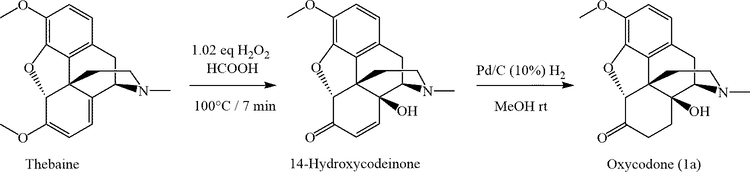

14-Hydroxycodeinone: This compound was prepared according to a modified literature procedure (A. Mata, D. Cantillo, C. O. Kappe, Eur. J. Org. Chem. 2017, 24, 6505-6510). In a 30 mL microwave vial equipped with a magnetic stir bar, thebaine (3.11 g, 10 mmol) was dissolved in 10 mL of formic acid under stirring. When the solid was fully dissolved (5-10 min stirring), the mixture was cooled to 5° C. using an ice/water bath. Then, 1.05 mL of 30% w/w H₂O₂ (1.02 equiv) was added under stirring and the mixture was heated in a microwave reactor at 100° C. for 7 min. The reaction mixture was cooled to room temperature using compressed air and then the solvent was evaporated under reduced pressure. The solid residue (which could be directly used for the next step) was dissolved in the minimum possible amount of saturated aqueous NaHCO₃ and extracted with CHCl₃ (3 × 50 mL). The combined organic layers there dried over MgSO₄ and dried under reduced pressure, yielding the title compound as brown crystals (83%).

Oxycodone (1a): This compound was prepared according to a modified literature procedure (A. Mata, D. Cantillo, C. O. Kappe, Eur. J. Org. Chem. 2017, 24, 6505-6510). 14-Hydroxycodeinone (10 mmol) was dissolved in 50 mL of HPLC grade methanol. 10% Pd/C (106 mg, 1 mol%) was added, and the resulting suspension was stirred under an atmosphere of hydrogen (1 atm, room temperature). The reaction progress was monitored by HPLC. Additional fresh 10% Pd/C was added if the reaction stopped before full conversion had been achieved. Upon completion, the crude reaction mixture was filtered through a plug of celite. The celite was washed with chloroform and the combined solutions were evaporated under reduced pressure to dryness. The residue was dissolved in chloroform (50 mL) and washed with saturated aqueous NaHCO₃. The organic layer was dried over Na₂SO₄ and evaporated to dryness. The resulting brown solid was recrystallized from ethanol/ethyl acetate 1:1, yielding oxycodone 1a as colorless needles (1984 mg, 63% over two steps).

### 2. Preparation of 14-Acetyloxycodone (1b)

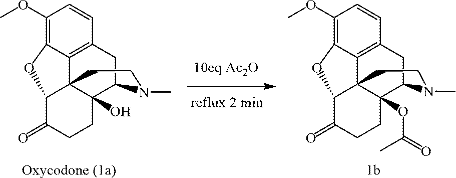

This compound was prepared according to a modified literature procedure (C.-Y. Cheng, L.-W. Hsin, Y.-P. Lin, P.-L. Tao, T.-T. Jong, Bioorg. Med. Chem. 1996, 4, 73-80). Oxycodone 1a (630 mg, 2 mmol) was placed in a round bottom flask and dissolved in 1.89 mL of acetic anhydride (20 mmol, 10 equiv) under gentle heating. The solution was then heated under reflux for ca. 2 minutes and left cooling to ambient temperature. The title compound crystallized after standing overnight at 6° C. (if the product does not crystallize, a small amount of diethyl ether can be added). The resulting crystals were collected by filtration and washed with cold diethyl ether to afford 636 mg (89%) of 1b as white needles.

### 3. Preparation of 14-Hydroxycodeinone O-acetyl Ester (1c)

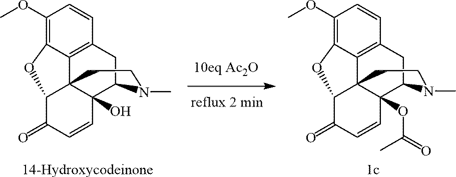

This compound was prepared according to a modified literature procedure (F. I. Carroll, C. G. Moreland, G. A. Brine, J. A. Kepler, J. Org. Chem. 1976, 41, 6, 996-1001). 14-Hydroxycodeinone (626 mg, 2 mmol) was placed in a round bottom flask and dissolved in 1.89 mL of acetic anhydride (20 mmol, 10 equiv) under gently heating. The solution was then heated under reflux for ca. 2 minutes and left cooling to ambient temperature. The title compound crystallized after standing overnight at 6° C. The resulting crystals were collected by filtration and washed with cold diethyl ether to afford 646 mg (91 % yield) of 1c as colorless crystals.

### 4. Preparation of Bis-O-diacetylmorphinone (1d)

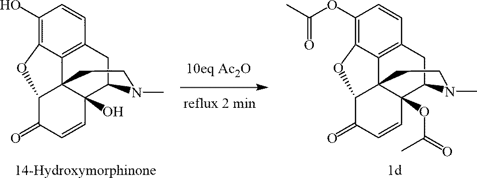

This compound was prepared according to a modified literature procedure (A. Machara, M. A. A. Endoma-Arias, I. Císařova, D. P. Cox, T. Hudlický, Synthesis 2016, 48, 1803-1813). 14-Hydroxymorphinone (594 mg, 2 mmol) was placed in a round bottom flask and dissolved in 1.89 mL of acetic anhydride (20 mmol, 10 equiv) under gentle heating. The solution was then heated under reflux for ca. 2 minutes and left cooling to ambient temperature. The title compound crystallized after standing overnight at 6° C. The resulting crystals were collected by filtration and washed with cold diethyl ether to afford 643 mg (84%) of 1 d as colorless crystals.

### 5. Synthesis of 6-Oxycodol (1e),

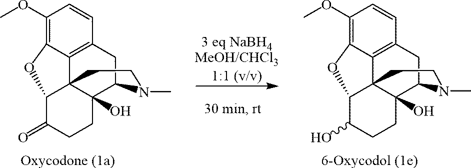

This compound was prepared according to a modified literature procedure (A. C. Currie, G. T. Newbold, F. S. Spring, J. Chem. Soc. 1961, 4693-4700). Sodium borohydride (226 mg, 6 mmol, 3 equiv) was added portionwise to a solution of oxycodone (630 mg, 2 mmol) in 30 mL of chloroform/methanol 1:1 at 10° C. After the addition was completed, the reaction mixture was stirred at room temperature for further 30 min. Then, the reaction was quenched with a large excess of a saturated solution of ammonium chloride in water. The solution was extracted with chloroform (3 × 50 mL). The combined organic layers were combined, dried over Na₂SO₄ and evaporated under reduced pressure. The resulting white solid was recrystallized from toluene/cyclohexane affording 361 mg (57%) of 6-oxycodol (1e) as colorless crystals.

II) Next, experimental procedures for electrochemical reactions in batch mode (A and B) and in a continuous mode using a flow cell (C) are described in the following.

### (A) General Procedure 1 for the Electrochemical Oxazolidination of 14-hydroxy Opioids and the Demethylative O,N-acetyl Transfer of O-acetyl Protected Derivatives in Batch Mode

In a 5 mL IKA ElectraSyn vial equipped with a stir bar, 0.15 mmol of the corresponding opioid precursor 1 were dissolved in 3 mL of a 0.1 M solution of tetraethylammonium tetrafluoroborate (Et₄NBF₄) in acetonitrile/methanol 4:1. After assembly of the electrochemical cell, equipped with a standard IKA graphite anode and a IKA stainless steel cathode, the solution was electrolyzed under a constant current of 5 mA until 2.4 F/mol had been passed. The cell voltage was in the range of 3.5 V to 5.0 V during the electrolysis process. After completion of the reaction, the reaction mixture was evaporated under reduced pressure to half of its original volume. The remaining solution was added to 500 mg of neutral alumina and filled into a short chromatography column and subsequently eluted with a suitable solvent (vide infra).

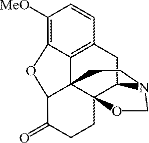 [SMILES: COc1ccc2c3c1OC1C(=O)CC[C@]45OCN(CC[C@]314)[C@@H]5C2]

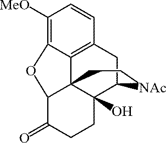

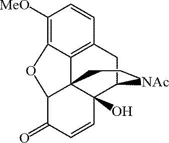

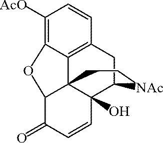

(5aR,6R,8aS,8a1S,11aR)-2-Methoxy-5,5a,9,10-tetrahydro-7H-6,8al-ethano-furo [2′,3′,4′,5′:4,5]phenanthro[9,8a-d]oxazol-11(11aH)-one (2a):

Following the general electrochemical reaction procedure 1 using oxycodone 1a (0.15 mmol, 47 mg) as the substrate and using a mixture of toluene/cyclohexane/chloroform 1:2:1 with 5% of methanol as eluent for column chromatography, 2a (41 mg, 89%) was obtained as a brown solid.

3-Methoxy-14-hydroxy-17-acetyl-4,5alpha-epoxymorphinan-6-one (2b):

Following the general electrochemical reaction procedure 1 using oxycodone-14-acetate (1b) (57 mg, 0.15 mmol) as the starting material and cyclohexane/ethyl acetate 1:3 with 5% methanol as eluent for column chromatography, 41 mg of the title compound, containing 5% w/w Et₄NBF₄ (NMR analysis), was isolated (75% purity-corrected yield).

3-Methoxy-14-hydroxy-17-acetyl-4,5alpha-epoxy-7,8-didehydro-morphinan-6-one (2c):

Following the general electrolysis procedure 1 using 14-acetyl codeinone 1c (57 mg, 0.15 mmol) as the substrate and cyclohexane/ethyl acetate 1:3 with 5% methanol as eluent for column chromatography, 38 mg (75%) of the title compound were isolated.

(4R,4aS,7aR,12bS)-3-Acetyl-4a-hydroxy-7-oxo-2,3,4,4a,7,7a-hexahydro-1H-4,12-methanobenzofuro[3,2-e]isoquinolin-9-yl acetate (2d):

Following the general electrolysis procedure 1 using 3,14-diacetyl morphinone 1d (57 mg, 0.15 mmol) as the substrate and ethyl acetate/cyclohexane/chloroform 6:2:1 with 5% methanol as eluent for column chromatography, 43 mg (78%) of the title compound were isolated.

### (B) General Procedure 2 for the Electrochemical Oxazolidination of 14-hydroxy Opioids in Batch Mode

In a 5 mL IKA ElectraSyn vial equipped with a stir bar, 0.60 mmol of the corresponding opioid precursor 1 were dissolved in 3 mL of a 0.1 M solution of potassium acetate (KOAc) in ethanol. After assembly of the electrochemical cell, equipped with an impervious graphite anode and a stainless steel cathode, the solution was electrolyzed under a constant current of 5 mA until 4 F/mol had been passed. The cell voltage was in the range of 3.5 V to 5 V during the electrolysis process. After completion of the reaction, the reaction mixture was evaporated under reduced pressure and the solid residue washed with cold water to remove the remaining KOAc.

(5aR,6R,8aS,8a1S,11aR)-2-Methoxy-5,5a,9,10-tetrahydro-7H-6,8a1-ethano-furo [2′,3′,4′,5′:4,5]phenanthro[9,8a-d]oxazol-11(11aH)-one (2a):

Following the general electrochemical reaction procedure 2 using oxycodone 1a (0.60 mmol, 189 mg) as the substrate, 2a (184 mg, 98%) was obtained as a brown solid.

### (C) Electrolysis of Oxycodone (1a) Using a Flow Cell

The setup depicted in FIG. 2 was utilized. A solution containing oxycodone (1a) (0.5 mmol) in 10 mL of 0.1 M Et₄NBF₄ in MeCN/MeOH 4:1 was pumped through the empty cell using a syringe pump with a flow rate of 2 mL/min, while being stirred with a magnetic stir bar. The outlet of the flow cell was returned to the reaction solution container, thus recirculating the mixture. When the system was stable and all air bubbles were displaced from the flow cell, the electrical power supply of the electrolysis cell was turned on under a constant current of 10 mA. After 2.4 F/mol of current had been applied, the power supply was turned off. Then, the inlet of the pump was taken out of the reaction mixture. Air was pumped through the cell until all the remaining reaction mixture had been collected from the cell output. The reaction mixture was then evaporated under reduced pressure to one third of its original volume. The remaining solution was added to 500 mg of neutral alumina and placed into a short chromatography column and eluted with toluene/cyclohexane/chloroform 1:2:1 with 5% of methanol. Evaporation of the solvent gave 124 mg of the oxazolidine 2a (79%).

### One-Pot Electrolysis/Hydrolysis Sequence for the Generation of Nor-Derivatives Using the Flow Electrolysis Method (C)

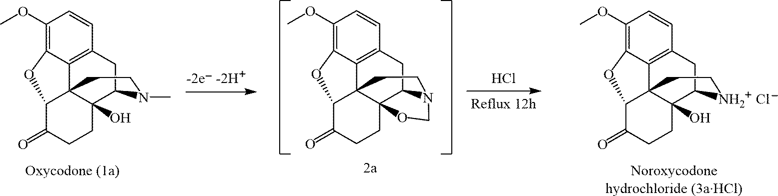

The flow electrolysis procedure described above was followed. When the electrolysis had been completed and all the solution had been collected in the solution reservoir, the crude reaction mixture was treated with 10 mL of 2 M HCl. The solution was heated under reflux overnight and then evaporated under reduced pressure. The solid residue was dissolved in water and washed with chloroform (30 mL). The aqueous phase was neutralized with saturated NaHCO₃ and extracted with chloroform (3 × 50 mL). The combined organic layers were combined, dried over Na₂SO₄ and evaporated under reduced pressure. The solid residue was dissolved in diethyl ether, and the solution sparged with HCI gas. Noroxycodone hydrochloride (3a-HCI) crystallized as a white powder (126 mg, 75% overall yield with respect to the initial oxycodone).

### One-Pot Electrolysis/Hydrolysis Sequence for the Generation of Nor-Derivatives Using the Batch Electrolysis Method 2 (B)

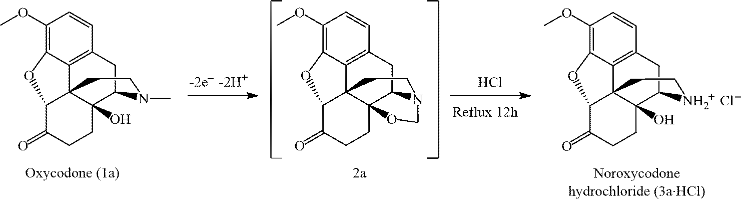

The general procedure 2 for the batch electrolysis described above was followed. When the electrolysis of 1a had been completed the solvent was evaporated under reduced pressure. The residue was treated with 10 mL of 2 M HCI. Then, the solution was heated under reflux for 20 min and evaporated under reduced pressure. The white powder obtained consisted of noroxycodone hydrochloride (3a•HCI) (94% essay yield) and potassium chloride.

III) The electrochemical conditions were varied and optimized using the example of an oxazolidination of oxycodone (1a)

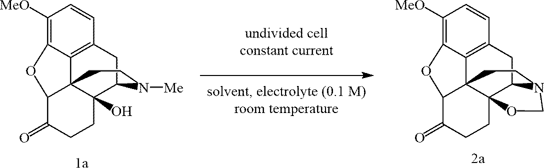

The results are shown in Tables 1 and 2 below:

TABLE 1

| Conditions^{a} | Conversion (%)^{b} | Selectivity (%)^{c} |
|---|---|---|
| MeCN. LiClO₄, (+)C/Fe(-), 5 mA. 2 F/mol | 29 | 90 |
| MeCN, LiBF₄, (+)C/Fe(-), 5 mA, 2 F/mol | 33 | 88 |
| MeCN, LiPF₆, (+)C/Fe(-), 5 mA, 2 F/mol | 17 | 82 |
| MeCN, NaCIO₄, (+)C/Fe(-), 5 mA, 2 F/mol | 47 | 89 |
| MeCN. Et₄NBF₄, (+)C/Fe(-), 5 mA, 2 F/mol | 79 | 96 |
| MeCN, nBu₄NBF₄, (+)C/Fe(-), 5 mA, 2 F/mol | 75 | 93 |
| MeCN, nBu₄NPF₆, (+)C/Fe(-), 5 mA, 2 F/mol | 76 | 96 |
| DMF. nBu₄NBF₄, (+)C/Fe(-), 5 mA, 2 F/mol | 74 | 70 |
| DMA, nBu₄NBF₄, (+)C/Fc(-), 5 mA, 2 F/mol | 80 | 31 |
| MeOH. nBu₄NBF₄, (+)C/Fe(-), 5 mA. 2 F/mol | 66 | 92 |
| nPrOH, nBu₄NBF₄, (+)C/Fe(-), 5 mA, 2 F/mol | 31 | 29 |
| MeOH/HFIP 4:1, nBu₄NBF₄, (+)C/Fe(-), 5 mA, 2 F/mol | 81 | 91 |
| CHCl₃/MeOH 3:1, nBu₄NBF₄, (+)C/Fe(-), 5 mA, 2 F/mol | 57 | 93 |
| CHCl₃/MeOH 1:1, nBu₄NBF₄, (+)C/Fe(-), 5 mA, 2 F/mol | 49 | 94 |
| MeCN/MeOH 4:1. nBu₄NBF₄, (+)C/Fe(-), 5 mA, 2 F/mol | 88 | 94 |
| MeCN/MeOH 9:1, nBu₄NBF₄, (+)C/Fe(-), 5 mA, 2 F/mol | 78 | 90 |
| MeCN/MeOH 4:1, nBu₄NBF₄, (+)Pt/Fe(-). 5 mA, 2 F/mol | 61 | 94 |
| MeCN/MeOH 4:1, nBu₄NBF₄, (+)RVC/Fe(-). 5 mA, 2 F/mol | 90 | 94 |
| MeCN/MeOH 4:1, nBu₄NBF₄, (+)C/Pt(-), 5 mA, 2 F/mol | 92 | 93 |
| MeCN/MeOH 4:1. nBu₄NBF₄, (+)C/C(-), 5 mA, 2 F/mol | 78 | 92 |
| MeCN/MeOH 4:1, nBu₄NBF₄, (+)C/Ni(-), 5 mA, 2 F/mol | 91 | 93 |
| MeCN/MeOH 4:1, nBu₄NBF₄, (+)C/Fe(-), 10 mA, 2 F/mol | 75 | 94 |
| MeCN/MeOH 4:1, nBu₄NBF₄, (+)C/Fe(-), 15 mA, 2 F/mol | 71 | 95 |
| MeCN/MeOH 4:1, nBu₄NBF₄, (+)C/Fe(-), 20 mA, 2 F/mol | 66 | 92 |
| MeCN/MeOH 4:1, Et₄NBF₄, (+)C/Fe(-), 10 mA, 2.4 F/mol | 89 | 94 |

^{a} General conditions: undivided cell; 0.15 mmol substrate in 3 mL solvent; 0.1 M supporting electrolyte (unless otherwise noted); 5 mL IKA Electrasyn vial; (+)C: graphite anode; Fc(-): stainless steel cathode.
^{b} Determined by HPLC peak area percent (205 nm).
^{c} Percent of product with respect to all peaks except the substrate (HPLC peak area percent, 205 nm).

TABLE 2

| Conditions^{a} | Conversion (%)^{b} | Selectivity (%)^{c} |
|---|---|---|
| MeCN/MeOH 4:1, Et₄NBF₄, (+)Cᵢₘₚ/Fe(-), 5 mA, 2.4 F/mol | 93 | 96 |
| MeCN/MeOH 4:1, Et₄NBF₄, (+)Cᵢₘₚ/Ni(-), 5 mA, 2.4 F/mol | 93 | 91 |
| MeCN/MeOH 4:1, Et₄NBF₄, (+)Cᵢₘₚ/Pt(-), 5 mA, 2.4 F/mol | 97 | 93 |
| MeCN/MeOH 4:1, Et₄NBF₄, (+)Cᵢₘₚ/Cᵢₘₚ(-), 5 mA, 2.4 F/mol | 84 | 95 |
| MeCN/H₂O 40:1, Et₄NBF₄, (+)Cᵢₘₚ/Fe(-), 5 mA, 2.4 F/mol | 70 | 90 |
| THF/H₂O 10:1, Et₄NBF₄, (+)Cᵢₘₚ/Fe(-), 5 mA, 2.4 F/mol | 85 | 93 |
| DME/H₂O 40:1, Et₄NBF₄, (+)Cᵢₘₚ/Fe(-), 5 mA, 2.4 F/mol | 60 | 99 |
| Acetone/H₂O 40:1, Et₄NBF₄, (+)Cᵢₘₚ/Fe(-), 5 mA, 2.4 F/mol | 76 | 92 |
| HFIP, Et₄NBF₄, (+)Cᵢₘₚ/Fe(-), 5 mA, 2.4 F/mol | 93 | 83 |
| DCM, Et₄NBF₄, (+)Cᵢₘₚ/Fe(-), 5 mA. 2.4 F/mol | 70 | 99 |
| MeCN/MeOH 4:1, KOAc, (+)Cᵢₘₚ/Fe(-), 5 mA, 2.0 F/mol | 67 | 99 |
| MeCN/MeOH 4:1, KOAc, (+)Cᵢₘₚ/Fe(-), 5 mA, 2.4 F/mol | 87 | 99 |
| MeCN/MeOH 4:1, KOAc, (+)Cᵢₘₚ/Fe(-), 5 mA, 2.8 F/mol | 93 | 97 |
| MeOH. KOAc, (+)CᵢₘₚFe(-), 5 mA, 2.4 F/mol | 88 | 78 |
| EtOH, KOAc, (+)Cᵢₘₚ/Fe(-), 5 mA, 2.4 F/mol | 80 | 99 |
| EtOH, KOAc, (+)Cᵢₘₚ/Fe(-), 5 mA, 3.0 F/mol | 97 | 99 |
| EtOH, 0.05 M KOAc, (+)Cᵢₘₚ/Fe(-), 5 mA, 2.4 F/mol | 80 | 99 |
| EtOH, 0.05 M KOAc, (+)Cᵢₘₚ/Fe(-), 5 mA, 3.0 F/mol | 97 | 99 |
| EtOH, 0.1 M 1a, 0.1 M KOAc, (+)Cᵢₘₚ/Fe(-), 5 mA, 3.0 F/mol | 98 | 99 |
| EtOH, 0.2 M 1a, 0.1 M KOAc, (+)Cᵢₘₚ/Fe(-), 5 mA, 3.0 F/mol | 86 | 99 |
| EtOH, 0.2 M 1a, 0.1 M KOAc, (+)Cimp/Fe(-), 5 mA. 4.0 F/mol | 98 | 99 |

^{a}General conditions: undivided cell; 0.15 mmol substrate (unless otherwise stated) in 3 mL solvent; 0.1 M supporting electrolyte (unless otherwise noted); 5 mL IKA Electrasyn vial; (+)C: graphite anode; (+)Cᵢₘₚ: impervious graphite anode; Fe(-): stainless steel cathode.
^{b}Determined by HPLC peak area percent (205 nm).
^{c}Percent of product and its N-formyl derivative with respect to all peaks except the substrate (HPLC peak area percent, 205 nm).

As evident from the results shown in Tables 1 and 2, a highly efficient and selective conversion of an opioid precursor compound to an oxazolidine intermediate may be achieved by electrolytic oxidation, which oxazolidine intermediate may then be hydrolysed to the respective nor-opioid compound.

As further evident from the results shown in Tables 1 and 2, the utilization of either quaternary ammonium or potassium salts had a significant beneficial influence on the reaction compared with in particular lithium salt electrolytes. The poorer performance of the lithium salt could be ascribed to the formation of a complex with the tertiary amine. The addition of protic solvents had a positive effect, providing a source of protons for the concurrent cathodic reduction. Although two protons are released during the formation of the iminium cation intermediate, a protic solvent clearly facilitates their transport and enhances the cathodic reduction. The utilization of pure methanol as solvent resulted in a lower conversion than the utilization of solvent mixtures comprising methanol. A combination of ethanol as the solvent and potassium acetate as the electrolyte provided the best results. Several electrode materials were also evaluated. None of the electrode combinations provided significant improvements with respect to the low-cost material combination of graphite or impervious graphite/stainless steel. Indeed, utilization of platinum as anode material, for example, resulted in lower conversion under otherwise identical conditions. Excellent results were achieved by applying a 20% excess of electricity (2.4 F/mol) under a current of 5 mA in MeCN/MeOH with Et₄NBF₄ as the supporting electrolyte (last entry of Table 1). The best results were achieved by applying an excess of electricity (3 or 4 F/mol) under a current of 5 mA in EtOH with KOAc as the supporting electrolyte (last two entries of Table 2), with nearly quantitative yield of the product obtained.

While the present disclosure has been described in detail by way of specific embodiments and examples, the disclosure is not limited thereto and various alterations and modifications are possible, without departing from the scope of the disclosure.

1-15. (canceled)

16. A process for preparing a compound of Formula (I) 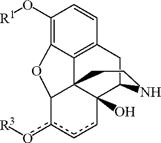
wherein
each 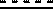
represents a single or double bond, provided that two double bonds are not adjacent to each other;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group;
R³ is selected from the group consisting of C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group or is absent;
wherein one or more hydrogen atoms on the R¹ and R³ groups may be replaced with F and/or Cl;
comprising the steps of
providing a compound of Formula (II) 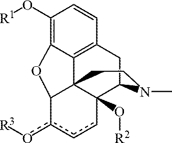
wherein
R¹, R³ and 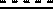
are as defined above; and
R² is selected from the group consisting of H, C(O)R⁶, S(O)R⁶,SO₂R⁶, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅-₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl; and
electrochemically demethylating the compound of Formula (II).

17. The process according to claim 16, wherein the step of electrochemically demethylating the compound of Formula (II) comprises an electrolytic oxidation of the tertiary N-methylamine functional group of the compound of Formula (II) and subsequently treating a thus obtained intermediate with an acid.

18. The process according to claim 17, wherein the intermediate is selected from the group consisting of a compound of Formula (III) and a compound of Formula (IV): 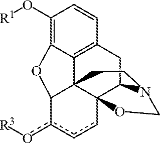
wherein R¹, R³ and 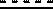
are as defined above; 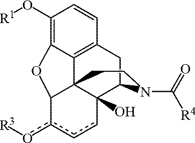
wherein
R¹, R³ and 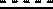
are as defined above and
R⁴ is selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅₋₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl.

19. The process according to claim 16, wherein the step of electrochemically demethylating the compound of Formula (II) comprises an electrolytic oxidation of the tertiary N-methylamine functional group of the compound of Formula (II) by means of an electrolytic unit comprising at least two electrodes and an electrolyte.

20. The process according to claim 19, wherein the electrolytic unit comprises an anode and a cathode, wherein the tertiary N-methylamine functional group of the compound of Formula (II) is electrolytically oxidized at the anode.

21. The process according to claim 20, wherein the anode comprises at least one of the group consisting of a carbon-containing material, and platinum.

22. The process according to claim 19, wherein the electrolyte is selected from the group consisting of a quaternary ammonium salt, a lithium salt, a sodium salt, a potassium salt and mixtures thereof.

23. The process according to claim 19, wherein the electrolytic unit further comprises a solvent.

24. The process according to claim 23, wherein the solvent is selected from the group consisting of acetonitrile, dimethylformamide, dimethylacetamide, methanol, ethanol, n-propanol, isopropanol, hexafluoroisopropanol (HFIP), trichloromethane, dichloromethane, tetrahydrofuran, methyltetrahydrofuran, acetone and mixtures thereof.

25. The process according to claim 16, wherein the step of electrochemically demethylating the compound of Formula (II) comprises an electrolytic oxidation of the tertiary N-methylamine functional group of the compound of Formula (II) in a batchwise manner.

26. The process according to claim 16, wherein the step of electrochemically demethylating the compound of Formula (II) comprises an electrolytic oxidation of the tertiary N-methylamine functional group of the compound of Formula (II) in a continuous manner, in particular using a flow cell.

27. The process according to claim 17, wherein the acid is selected from the group consisting of hydrochloric acid, acetic acid and sulfuric acid.

28. The process according to claim 16,
wherein the compound of Formula (I) is a compound of Formula (Ia), 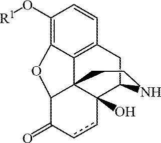
wherein 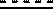
represents a single or double bond;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group, wherein one or more hydrogen atoms on the R¹ groups may be replaced with F and/or CI; and
wherein the compound of Formula (II) is a compound of Formula (IIa), 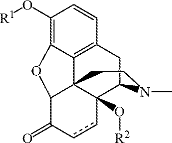
wherein
R¹ and 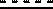
are as defined above; and
R² is selected from the group consisting of H, C(O)R⁶, S(O)R⁶,SO₂R⁶, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅-₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl.

29. A process for preparing a compound of Formula (V) 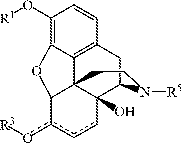
wherein
each 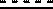
represents a single or double bond, provided that two double bonds are not adjacent to each other;
R¹ is selected from the group consisting of H, C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group;
R³ is selected from the group consisting of C₁₋₁₀ alkyl, C₆₋₁₀ aryl, C₃₋₁₀ cycloalkyl, C₁₋₁₀ alkylene-C₆₋₁₀ aryl, C₁₋₁₀ alkylene-C₃₋₁₀ cycloalkyl and a protecting group or is absent;
R⁵ is selected from the group consisting of C₁₋₁₀ alkyl, C₂₋₁₀ alkenyl, C₆₋₁₀ aryl, C₃₋₁₀ cycloalkyl, C₃₋₁₀ cycloalkenyl, C₁₋₁₀ alkylene-C₆₋₁₀ aryl, C₁₋₁₀ alkylene-C₃₋₁₀ cycloalkyl;
wherein one or more hydrogen atoms on the R¹, R³ and R⁵ groups may be replaced with F and/or Cl;
comprising the steps of
providing a compound of Formula (II) 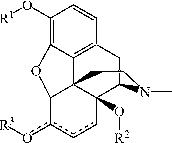
wherein
R¹, R³ and 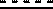
are as defined above; and
R² is selected from the group consisting of H, C(O)R⁶, S(O)R⁶,SO₂R6, P(O)R⁶R⁷, P(O)(OR⁶)R⁷, and P(O)(OR⁶)(OR⁷), and
R⁶ and R⁷ are each independently selected from the group consisting of C₃₋₁₀ cycloalkyl, C₃₋₁₀ heterocycloalkyl, C₃₋₁₀ cycloalkenyl, C₁₋₁₀ alkyl, C₂₋₁₀ alkenyl, C₆₋₁₀ aryl and C₅-₁₀ heteroaryl, each of the groups being unsubstituted or substituted with one or more substituents independently selected from C₁₋₄ alkyl, O-C₁₋₄ alkyl, halogen, CN, NO₂, C₆₋₁₀ aryl and O-C₆₋₁₀ aryl;
electrochemically demethylating the compound of Formula (II) to yield a compound of Formula (I) 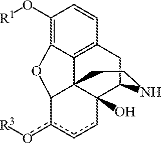
wherein R¹, R³ and 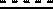
are as defined above; and
subsequently reacting the compound of Formula (I) with a compound of Formula (VI) 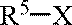
wherein R⁵ is as defined above and X represents a leaving group, in the presence of a base.

30. The process according to claim 29, wherein the compound of Formula (V) is selected from the group consisting of naloxone, naltrexone and nalbuphine.

31. The process according to claim 20, wherein the anode comprises at least one of the group consisting of graphite, impervious graphite, reticulated vitreous carbon, glassy carbon, carbon felt, and boron-doped diamond.

32. The process according to claim 20, wherein the cathode comprises at least one of the group consisting of an iron-containing material, a nickel-containing material, platinum, lead, mercury and a carbon-containing material.

33. The process according to claim 20, wherein the cathode comprises at least one of the group consisting of stainless steel, graphite, reticulated vitreous carbon, glassy carbon, carbon felt, and boron-doped diamond.

34. The process according to claim 23, wherein the solvent is a protic solvent.
